(19) United States
(12) Patent Application Publication
(10) Pub. No.: US 2025/0122139 A1
(43) Pub. Date: Apr. 17, 2025

(54) MICROPOROUS CATALYSTS FOR DIRECT SYNTHESIS OF BRANCHED ENALS WITH HIGH SELECTIVITY

(71) Applicant: BOARD OF REGENTS, THE UNIVERSITY OF TEXAS SYSTEM, Austin, TX (US)

(72) Inventor: Simon M. HUMPHREY, Lakeway, TX (US)

(73) Assignee: BOARD OF REGENTS, THE UNIVERSITY OF TEXAS SYSTEM, Austin, TX (US)

(21) Appl. No.: 18/919,076

(22) Filed: Oct. 17, 2024

Related U.S. Application Data
(60) Provisional application No. 63/591,007, filed on Oct. 17, 2023.

(51) Int. Cl. C07C 45/50; B01J 31/16; B01J 31/18; B01J 31/20; B01J 31/24; B01J 37/00; B01J 37/03; B01J 37/06

(52) U.S. Cl. CPC C07C 45/505; B01J 31/1691; B01J 31/181; B01J 31/20; B01J 31/24; B01J 37/009; B01J 37/03; B01J 37/06; B01J 2231/321; B01J 2531/004; B01J 2531/0205; B01J 2531/822; B01J 2531/827; B01J 2531/828

(57) ABSTRACT

Disclosed herein are new metallated MOF catalysts, preferably, Rh-PCM-101 or Rh-AsCM-102, as well of methods using such catalysts in hydroformylation reactions and in tandem hydroformylation/enolization reactions. In a specific embodiment, a metallated phosphine MOF catalyst according to the disclosure, preferably Rh-PCM-101, is used in a single-step hydroformylation process to transform propene into 2-ethyl-2-hexenal.

### CROSS-REFERENCE TO RELATED APPLICATION

This application claims the benefit of Provisional Application Ser. No. 63/591,007, filed Oct. 17, 2023, which is herein incorporated by reference.

### GOVERNMENT SUPPORT

This invention was made with government support under Grant No. DE-SC0021128 awarded by the Department of Energy. The government has certain rights in the invention.

### FIELD OF THE INVENTION

The present invention relates generally to the field of heterogenous microporous catalysts useful for hydroformylation and other chemical reactions, including the direct synthesis of branched enals with high selectivity.

### BACKGROUND OF THE INVENTION

Hydroformylation (HFM) represents one of the world's largest volume industrial processes for the direct conversion of olefins (alkenes, arenes) into aldehydes by reaction with 1 equivalent CO and 1 equivalent H₂ (commonly referred to as syngas). Major chemical manufacturers, including BASF (oxo process), RDS, Exxon, Union Carbide, Celanese, LyondellBasell, have large scale plants operating with Rh and/or Ir-based homogeneous catalysts (sometimes referred to as molecular catalysts). Estimates place the world annual output of aldehydes produced by these processes at approximately 10 MM metric tons with approximately 25% of that output coming from the Houston, Texas area.

Aldehydes produced by HFM have a multitude of uses. One of highest volume products produced by HFM of propene is the C4-molecule butyraldehyde also known as 1-butanal. 1-butanal is often further processed to manufacture other useful chemicals. For example, 1-butanal may be further subjected to direct hydrogenation to produce n-butanol and other synthetic linear alcohols and acids. As another example, 1-butanal may be subjected to acid or base-catalyzed aldol condensation to give branched C8 intermediates, which are subsequently dehydrated to yield the corresponding enal (2-ethyl-2-hexenal), which may be hydrogenated in another step to yield 2-ethylhexanol (2-EH). Approximately 2 MM metric tons of 2-EH are produced worldwide per year and used to manufacture lubricants, surfactants, detergents, emollients, and plasticizers.

One of the problems with the prior art multi-step process is that the steps cannot all be performed in the same reactor (i.e., the process requires isolation at the intermediate stages), which increases the manufacturing equipment required, and accordingly, increases the cost of production. In the first reaction, molecular catalysis enables aldehyde formation. The catalyst must be removed from the aldehyde, the aldehyde must be purified, and then placed into a different reactor where acid or base-catalyzed condensation may occur to convert the aldehyde into an aldol. This is because the acid or base needed for the condensation reaction inadvertently kills molecular catalysts such as those used in the prior art. In other words, catalysis and condensation cannot occur in the same reactor in typical prior art hydroformylation reactions.

A zinc-based metallic organic framework (MOF) catalyst has been previously reported by Gaumann et al. (Tandem Hydroformylation-Aldol Condensation Reaction Enabled by Zn-MOF-74, Chem. Eur. J. 2023, 29, e202300939) and used in a cobalt-catalyzed hydroformylation of 1-hexene. However, the desired product was only produced as a minority product (at a maximum of about 30%), which is not high enough for industrial applications. In addition, the Gaumann reported catalyst cannot support Rh, which is currently the most industrially relevant metal for hydroformylation.

### SUMMARY OF THE INVENTION

The present disclosure describes metallated MOF catalysts that can work as microporous solid state ligands to overcome the limitations in the prior art. Specifically, metallated MOF catalysts, for example, Rh-PCM-101, Ir-PCM-101, Pt-PCM-101, and Rh-AsCM-102, may be used in single-step hydroformylation reactions to transform alkenes into other useful chemicals. These metallated MOF catalysts have active single site catalysts such as Rh(I)- or Ir(I)-based ligands that directly mimic currently used molecular homogenous catalysts, but the single site catalysts are protected by the metallic organic framework (MOF) inside well-defined micropores that protect the catalysts from degradation and render the catalysts insoluble (heterogenous), which enables simple catalyst-product separation. In addition, the physical structure of the catalysts inside the MOF micropores creates nano-sized reaction flasks that restrict reaction orientation. As a result, when used in HFM reactions, these catalysts primarily produce branched aldehydes. This is the opposite of current HFM reactions, which primarily produce linear products, and therefore these metallated MOF catalysts will be important for synthesizing otherwise hard-to-produce branched products.

In addition, subsequent to an HFM reaction of an olefin, the direct liquid products may be removed from the HFM reactor and the metallated MOF catalyst recovered by simple filtration or centrifugation. Alternatively, the metallated MOF catalyst may be removed from the reactor and thereby separated from the direct liquid products via a partition or a moving bed assembly. Adding aqueous sodium hydroxide, an organic base, or a Bronsted acid to the direct liquid products results in direct aldol condensation resulting in new enal products. This process works for smaller and larger alkene feedstocks, including C3-C8 alkenes and their corresponding cyclo-alkenes, such as propene (C3), butene (C4), pentene (C5), hexene (C6), cyclohexene, heptene (C7), octene and cyclooctene (C8).

In certain embodiments, a catalyst is a heterogenous catalyst comprised of a metal-organic framework and a 4d or 5d transition metal species contained in one or more active sites. The metal-organic framework may be a phosphine-based metal organic framework such as: PCM-101, PCM-102, or PCM-201. Alternatively, the metal-organic framework may be an arsine-based metal-organic framework such as AsCM-102, AsCM-201, or AsCM-303 [Zns(ClO₄)(TPZA)₄]³⁺.

In certain embodiments, the 4d or 5d transition metal species of the catalyst is one of Ru^{II}, Rh^{I}, Ir^{I}, Os, Pd^{II}, and Pt^{II} bearing common ligands.

A method for catalysis of olefins is also described. In one embodiment, the method comprises: (a) submerging a metallated MOF catalyst in a liquid solvent inside a reactor; (b) adding an olefin to the reactor; (c) maintaining a CO:H₂ partial pressure ratio in the reactor within a range of 1:1 and 3:1; (d) maintaining the temperature of the reactor between 55° C. and 85° C. for a reaction time; and (e) maintaining the overall pressure in the reactor between 40 and 55 bar during the reaction time, wherein the reaction time is between eleven and twenty-four hours. Preferably, the reaction time is seventeen hours.

In another embodiment, a method for catalysis of olefins comprises: (a) submerging a metallated MOF catalyst in a liquid solvent inside a reactor; (b) adding an olefin to the reactor; (c) maintaining a CO:H₂ partial pressure ratio in the reactor within a range of 1:1 and 3:1; (d) maintaining the temperature of the reactor between 55° C. and 85° C. for a reaction time; (e) maintaining the overall pressure in the reactor between 40 and 55 bar during the reaction time, wherein the reaction time is between eleven and twenty-four hours; (f) removing the metallated MOF catalyst; (g) adding one or more of aqueous sodium hydroxide, an organic base, or a Bronsted acid to the reactor; and (h) recovering an enal product. Preferably, the temperature for this catalysis method is 70° C. and/or the overall pressure is 40 bar. The aqueous sodium hydroxide, organic base, or Bronsted acid is added to a concentration sufficient to promote aldol condensation, preferably in a concentration within the range from 0.001M to 1M, preferably with a range of 0.001M to 1M, and most preferably, at a concentration of 0.01M.

In certain embodiments of the catalysis method, removing the metallated MOF catalyst comprises removing it from the reactor and/or separating it from the reaction components by filtering or centrifuging the metallated MOF catalyst. The metallated MOF catalysts according to the invention are microporous solid-state ligands and thus easily separated from the other reaction components (i.e., reactants), which are either liquid or gas.

In certain other embodiments of the catalysis method, the reactor is a moving bed reactor and removing the metallated MOF catalyst comprises using a moving bed assembly to remove the metallated MOF catalyst from one or more reactants before steps (g) and (h) are performed.

In other embodiments of the catalysis method, removing the metallated MOF catalyst comprises partitioning the metallated MOF catalyst away from the liquid solvent and one or more reaction products before steps (g) and (h) are performed. Such partitioning comprises dividing the internal space of the reactor into smaller, distinct sections or compartments, whether through physical barriers or internal design elements such that the metallated MOF catalyst remains in one section or compartment while the rest of the reaction components are in a different section or compartment to which one or more of aqueous sodium hydroxide, an organic base, or a Bronsted acid may then be added to cause enolization.

In certain embodiments of the catalysis method, the CO:H₂ partial pressure ratio is 1:1. In alternative embodiments, the CO:H₂ partial pressure ratio is 3:1. The CO:H₂ partial pressure ratio may be chosen within the range from 1:1 to 3:1 in order to promote one type of reaction product over another type of reaction product. The CO:H₂ partial pressure ratio impacts the reaction rate, as well as the selectivity between linear and branched reaction products.

In some embodiments, a metallated MOF catalyst is one of Rh-PCM-101, Ir-PCM-101, Pt-PCM-101, Rh-PCM-102, Ir-PCM-102, Pt-PCM-102, Rh-PCM-201, Ir-PCM-201, Pt-PCM-201, Rh-AsCM-102, Rh-AsCM-201, Rh-AsCM-303, Ir-AsCM-102, Ir-AsCM-201, Ir-AsCM-303, Pt-AsCM-102, Pt-AsCM-201, or Pt-AsCM-303. In alternative embodiments, two or more of Rh-PCM-101, Ir-PCM-101, Pt-PCM-101, Rh-PCM-102, Ir-PCM-102, Pt-PCM-102, Rh-PCM-201, Ir-PCM-201, Pt-PCM-201, Rh-AsCM-102, Rh-AsCM-201, Rh-AsCM-303, Ir-AsCM-102, Ir-AsCM-201, Ir-AsCM-303, Pt-AsCM-102, Pt-AsCM-201, or Pt-AsCM-303 may be used. In a preferred alternative embodiment, Rh-PCM-101 and Rh-AsCM-102 are used in a 1:1 ratio.

In certain embodiments, the olefin used in the catalysis method is one of: styrene, cyclobutene, cyclopropene, cyclohexene, cyclooctene, propene, butene, pentene, hexene, heptene and octene. In other embodiments, the olefin used in the catalysis method is another alpha-olefin, arene, diene, or polyene.

In a preferred embodiment, the catalysis method described herein uses Rh-PCM-101 as the metallated MOF catalyst, propene as the olefin, and the recovered enal product is 2-ethyl-2-hexenal.

In another preferred embodiment, the catalysis method described herein uses Rh-AsCM-102 as the metallated MOF catalyst, propene as the olefin, and the recovered enal product is 2-ethyl-2-hexenal.

### BRIEF DESCRIPTION OF THE FIGURES

FIG. 1 shows space-filling models of the periodic structure of PCM-101 (with Ni₃OH nodes) (101) in the conversion reaction using [RhCl(CO)₂]₂ to form the metallated phosphine MOF catalyst Rh-PCM-101 (102) and depicts the structure of the catalytic sites in the resulting Rh-PCM-101 (103) as well as the impact on those catalytic sites when under 40 bar pressure with only CO gas (104) or H₂ gas (105).

FIG. 2(a) shows space-filling models of the periodic structure of PCM-101 in the conversion reaction using IrCl(C₂H₄)₂ to form the metallated phosphine MOF catalyst Ir-PCM-101. FIG. 2(b) shows space-filling models of the periodic structure of PCM-101 in the conversion reaction using Pt(CH₃)₂Cl₂ to form the metallated phosphine MOF catalyst Pt-PCM-101.

FIG. 3 shows FT-IR spectra of Rh-PCM-101. Specifically, FIG. 3 shows the FT-IR spectra for CO only (301), Rh-PCM-101 post-catalysis (302), and Rh-PCM-101 (303).

FIG. 4 shows PXRD (powder x-ray diffraction) results of H₂ only (401), CO only (402), Rh-PCM-101 post-catalysis (403), and Rh-PCM-101 (404).

FIG. 5 shows GC-MS (gas chromatography mass spectrometry) product distribution results for the catalysis reactions of 1-hexene at CO:H₂ of 3:1 with Rh-PCM-101 as the catalyst (black bar), n-heptanal at CO:H2 of 3:1 with Rh-PCM-101 as the catalyst (hatched bars), and n-heptanal at CO:H₂ of 3:1 with no Rh-PCM-101 catalyst (white bars).

FIG. 6 shows the conversion of olefins to enals and branched alcohols via either the prior art multi-step process or direct tandem catalysis using one of the metallated MOF catalysts described herein.

FIG. 7 shows the structure of AsCM-303 with catalytic sites of metal ligands.

FIG. 8 shows the FT-IR spectra of Rh-PCM-101 charged with CO (901), Rh-PCM-101 post-catalysis (902), and Rh-PCM-101 (903).

FIG. 9 shows PXRD results for Rh-PCM-101 post-catalysis (1001), Rh-CO-PCM-101 (1002), and PCM-101 (1003).

FIG. 10 shows an HMF reaction of 1-hexene (1101), which may yield various products depending on the conditions including n-heptanal (1102), 2-methyl-hexanal (1103), and 2-ethyl-pentanal (1104).

FIG. 11 shows a graph with conversion percentage and TOF (turnover frequency) plotted against time for an HMF reaction of 1-Hexene using Rh-AsCM-102-Co as the catalyst at a partial pressure ratio of 1:1 for CO:H₂, 40 bar pressure, and 70° C.

FIG. 12 shows (a) an HFM reaction of 1-hexene (1301) using Rh-AsCM-102 as the catalyst to yield n-heptanal (1302) as the major product with 2-methyl-hexanal (1303) and 2-ethyl pentanal (1304) as minor products, (b) followed by enolization of the heptanal to yield (Z)-2-pentylidenenonanal (1305).

### DETAILED DESCRIPTION OF THE INVENTION

Unless defined otherwise, all scientific or technical terms used herein shall have the same meaning as is commonly understood by one of skill in the art. Unless specific definitions are provided, the nomenclature used in connection with, and the procedures and techniques of chemistry, including inorganic, organic, and physical chemistry described herein are those well-known and commonly used in the art. Standard techniques may be used for chemical synthesis and chemical analysis consistent with the additional disclosures provided herein.

### Definitions

As used in the specification and the claims, the singular forms “a,” “an,” and “the” include the plural unless the context clearly dictates otherwise.

Unless otherwise explicitly noted, where the disclosures uses the phrase “within a range,” it is meant to include the values given for the beginning and end of such range.

In the specification and the claims that follow, reference will be made to a number of terms, which shall be defined to have the following meanings:
2-EH is an acronym for the chemical compound 2-ethylhexanol.

Aldehyde refers to an organic compound containing a functional group with the structure R—CH═O. Common aldehydes include methanal (formaldehyde) and ethanal (acetylaldehyde).

AsCM is an acronym for arsine coordination materials. Specific arsine-based MOFs known in the art may be referred to by AsCM combined with a specific number, e.g. AsCM-102.

Catalyst has its plain and ordinary meaning; however, this disclosure uses the phrase “metallated MOF catalyst” to refer to the heterogeneous catalyst that consists of a MOF backbone (i.e., a solid micropore metal-organic framework structure) with one or more metal ligand catalysts each contained in a catalytic site. Similarly, “metallated phosphine MOF catalyst” refers to the heterogeneous catalyst that consists of a phospine-based MOF backbone, such as, but not limited to, PCM-101, PCM-102, or PCM-201, with one or more metal ligand catalysts each contained in a catalytic site. Likewise, “metallated arsine-based MOF catalyst” refers to the heterogeneous catalyst that consists of an arsine-based MOF backbone, such as, but not limited to, AsCM-102, AsCM-201, and AsCM-303, with one or more metal ligand catalysts each contained in a catalytic site.

COD is an acronym for cyclooctadiene.

DCM is an acronym for dichloromethane.

DMF is an acronym for dimethylformamide.

HFM is an acronym referring to hydroformylation.

MOF is an acronym referring to metal-organic framework.

Olefins refers to compounds made of carbon and hydrogen that contain at least one double bond between a pair of carbon atoms. Olefins includes alkenes and arenes.

PCM is an acronym for phosphine coordination materials. Specific phosphine-based MOFs known in the art may be referred to by PCM combined with a specific number, e.g. PCM-101.

Pnictogen refers to a chemical element or compound that is part of group 15 of the periodic table, such as nitrogen, phosphorus, arsenic, antimony, or bismuth.

SSL is an acronym referring to solid state ligand.

Syngas refers to synthesis gas, which is a mixture of carbon monoxide (CO) and hydrogen (H₂).

### Overview

The present disclosure describes new catalyst materials, including those prepared by post-synthetic modification of a phosphine-based MOF such as PCM-101, PCM-102, or PCM-201, or prepared using an arsine-based MOF, such as AsCM-102, AsCM-201, or AsCM-303, by installation of secondary low-valent metal species, specifically, 4d and 5d transition metals. These new catalyst materials are unique examples of MOF-supported catalysts that contain catalytic sites not accessible to conventional molecular chemistry. Secondary low-valent metal species useful for such catalysts include complexes based on Ru^{II}, Rh^{I}, Ir^{I}, Pd^{II}, and Pt^{II}, bearing common ligands including halides, CO, and/or alkyls.

The present disclosure also describes use of these new catalyst materials as hydroformylation catalysts, which are able to achieve different product outcomes than their molecular counterparts. For example, Rh-based PCM-101 or AsCM-102 catalysts according to the invention can achieve direct production of 2-ethyl-2-hexenal from propene in a single step (as opposed to three steps in the prior art process) with near 100% selectivity as a direct function of control over CO:H₂ gas partial pressures. Ideally in this direct tandem HFM/aldol condensation method, the CO:H₂ ratio is 1:1, the overall pressure is maintained in a range of 40-55 bar, preferably 40 bar, the reaction temperature is in a range between 55° C. and 85° C., preferably 70° C., the metallated MOF catalyst is removed from the reaction, and one or more of aqueous sodium hydroxide, an organic base, such as t-BuOK, or a Bronsted acid, such as ZSM-5, is then added at a concentration within the range from 0.001M to 1M, and most preferably, at a concentration of 0.01M in order to promote aldol condensation to the 2-ethyl-2-hexenal product.

Benefits of the catalysts described herein include: (i) catalyst enabled direct access to value-added products that exist within the current product chain but do not require isolation at each intermediate stage; (ii) unlike conventional homogeneous catalysts used in this filed, the catalysts described herein are heterogeneous microporous solid state ligands so catalyst recovery is greatly simplified; (iii) the same process works for lighter and heavier n-alkenes (for example, 1-hexene+CO+H₂ to yield directly the corresponding C-14 enals); and (iv) the MOF pores are hydrophobic in the catalysts of the invention, so the catalysts resist the presence of water, which is a potential poison to molecular catalysts.

### Post-Synthetic Metallation of MOFs to Form New Catalysts

Post-synthetic metallation of MOFs as solid-state ligands generates crystalline, heterogeneous catalyst materials with crystallographically-defined, single-site (i.e., homogeneous) active sites in unique microenvironments. This can be thought of as a scaffold-like structure to protect catalytic sites. Low-valent metal site-occupancy, also known as loading, in MOFs as solid state ligands (SSLs) can be controlled by post-synthetic metalation using specified molar amounts of low-valent 4d and 5d transition metal species. Examples of low-valent 4d and 5d transition metal species that may be used in post-synthetic metalation of MOFs include: [RhCl(CO)₂]2, [IrCl(C₂H₄)₂]₂, Pt(CH₃)₂Cl₂, Pt(COD)(CH₃)₂, and PdCl₂(COD).

Phosphine MOFs useful as backbones for the catalysts described herein include PCM-101, PCM-102, and PCM-201, which have been previously characterized. See, e.g., Humphrey et al., “A Metal-Organic Framework with Cooperative Phosphines That Permit Post-Synthetic Installation of Open Metal Sites,” Angewandte Chemie, 2018, 57, 30, pp. 9295-9299 (characterizing PCM-101); Humphrey et al., “Low-Valent Metal Ions as MOF Pillars: A New Route Toward Stable and Multifunctional MOFs,” J. Am. Chem. Soc., 2021, 143, 34, pp. 13710-13720 (characterizing PCM-102), each of which is herein incorporated by reference. Other MOFs useful as backbones for the catalysts described herein include arsine-based MOFs, including AsCM-102, AsCM-201, and AsCM-303 (Zns(ClO₄)(TPZA)₄). Metallated arsine-based MOF catalysts can have a higher activity than metallated phosphine MOF catalysts using the same metal. However, use of metallated phosphine MOF catalysts may be more industrial acceptable due to a perceived risk of arsenic toxicity if it were to leach from the metallated arsine-based catalysts. However, the scaffold structure imparted in the metallated arsine-based catalysts described herein appears to prevent leaching and greatly reduce any risk of toxicity.

In a reaction to form the metallated MOF catalyst, consideration is given to the number of coordination sites available in the MOF such that the molar amount of the transition metal species added does not result in a loading ratio above 1. While it is not necessary for every potential coordination site to include a transition metal species to act as a catalyst-meaning loading ratios above zero and below 1 are acceptable—a loading ratio above 1 indicates more transition metal species than coordination sites, and the excess metal may negatively impact the efficiency of catalysis. Stated another way, 100% coordination site occupancy is not required in the metallated MOF catalysts disclosed herein. The coordination site occupancy in the metallated MOF catalyst may be in a range from 1-100%, 10-100%, 20-100%, 30-100%, 40-100%, 50-100%, 60-100%, 70-100%, 80-100, or most preferably, 90-100%.

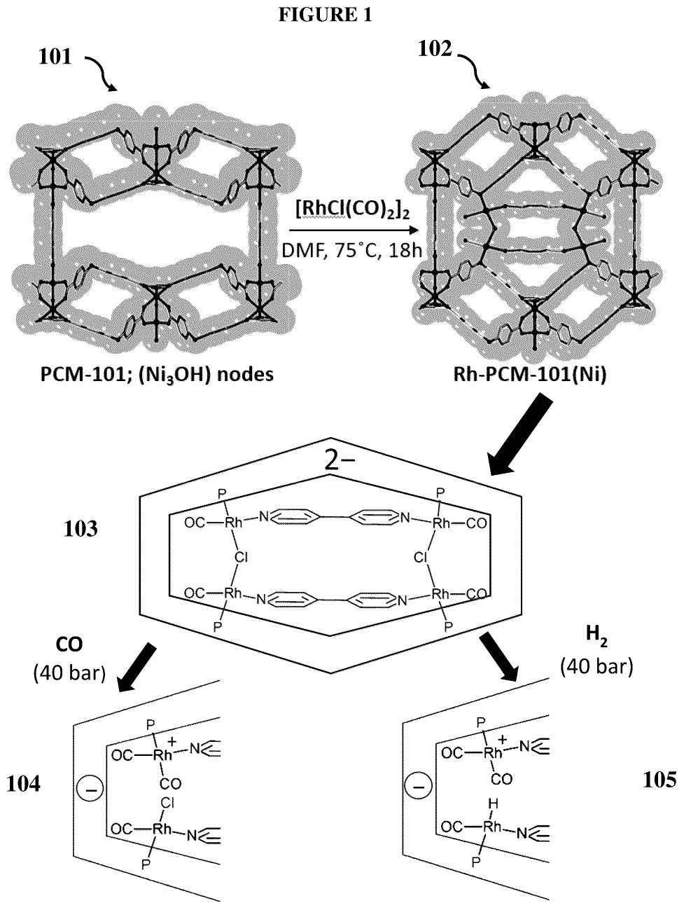

Examples of post-synthetic metalation of MOFs to form catalysts include incorporation of low-valent 4d and 5d transition metal species in trans-P2 binding pockets of the solid state ligand PCM-101 to form Rh-PCM-101, Ir-PCM-101, and Pt-PCM-101. FIG. 1 shows space-filling models of the periodic structure of PCM-101 (with Ni₃OH nodes) (101) in the conversion reaction using [RhCl(CO)₂]₂ to form the metallated phosphine MOF catalyst Rh-PCM-101 (102) and depicts the structure of the catalytic sites in the resulting Rh-PCM-101 (103) as well as the impact on those catalytic sites when under 40 bar pressure with only CO gas (104) or H₂ gas (105). To prepare Rh-PCM-101 (102) as shown in FIG. 1, [RhCl(CO₂)₂]₂ was added to PCM-101 with DMF as a solvent in a reaction at 75° C. that was allowed to continue for eighteen hours. The resulting metallated phosphine MOF catalyst Rh-PCM-101 is comprised of the PCM-101 backbone, which protects [RhCl]⁺ dimers inside the catalytic site(s) as shown. The structure of the catalytic sites during an HFM reaction may be slightly altered by controlling the partial pressures of CO gas (104) and H₂ gas (105) to facilitate recovery of specific products and change the reaction rate. The partial pressure ratio of CO:H₂ gas has been shown to change the selectivity of reaction products in an HFM reaction resulting in the ability to control whether linear or branched products are the major products.

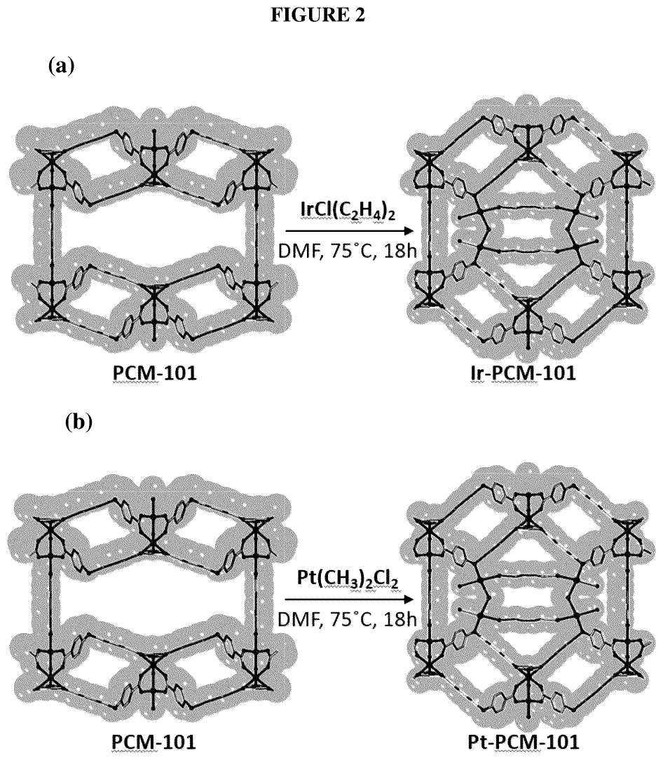

FIG. 2(a) shows space-filling models of the periodic structure of PCM-101 in the conversion reaction using IrCl(C₂H₄)₂ to form the metallated phosphine MOF catalyst Ir-PCM-101. That reaction was performed in the presence of DMF at 75° C. and allowed to continue for eighteen hours. FIG. 2(b) shows space-filling models of the periodic structure of PCM-101 in the conversion reaction using Pt(CH₃)₂Cl₂ to form the metallated phosphine MOF catalyst Pt-PCM-101. That reaction was also performed in the presence of DMF at 75° C. and allowed to continue for eighteen hours.

Low-valent 4d and 5d transition metals useful in post-synthetic metalation of MOFs to generate catalysts according to the disclosure herein include: ruthenium (Ru), rhodium (Rh), palladium (Pd), osmium (Os), iridium (Ir), and platinum (Pt). Preferred low-valent metal species useful for the metallated MOF catalysts disclosed herein include complexes based on Ru^{II}, Rh^{I}, Ir^{I}, Pd^{II}, and Pt^{II}, bearing common ligands including halides, CO, and/or alkyls. Examples of low-valent 4d and 5d transition metal species that may be used in post-synthetic metalation of MOFs include: [RhCl(CO)₂]₂, IrCl(C₂H₄)₂, Pt(CH₃)₂Cl₂, Pt(COD)(CH₃)₂, and PdCl₂(COD).

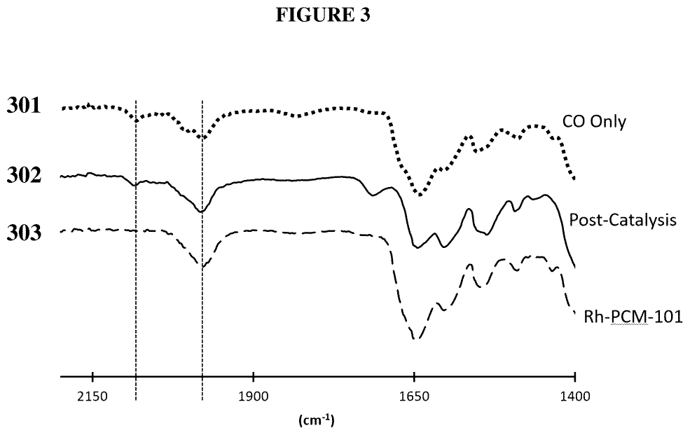

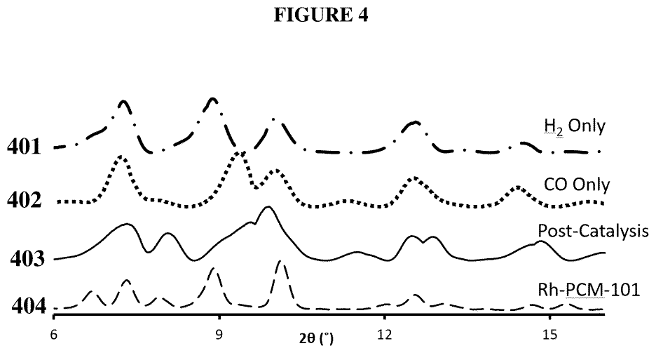

Metallated MOF catalysts according to the disclosure herein may be characterized by FT-IR spectra, as well as PXRD. FIG. 3 shows FT-IR spectra of Rh-PCM-101. Specifically, FIG. 3 shows the FT-IR spectra for CO only (301), Rh-PCM-101 post-catalysis (302), and Rh-PCM-101 (303). FIG. 4 shows PXRD (powder x-ray diffraction) results of H₂ only (401), CO only (402), Rh-PCM-101 post-catalysis (403), and Rh-PCM-101 (404). As demonstrated in FIG. 3 and FIG. 4, the structure of the active sites in Rh-PCM-101 are stable even after being used as a catalyst.

### Hydroformylation Reactions Using Metallated MOF Catalysts

Hydroformylation reactions using metallated MOF catalysts of the present invention may be performed by submerging the metallated MOF catalyst solid directly in an olefin (alkenes, arenes) in a Parr pressure reactor or other reactor that enables control of the overall pressure and the partial pressures of CO gas and H₂ gas. The solid state of the metallated MOF catalysts enables the ability to partition the catalyst in a reactor such that separation of the catalyst from the reaction product is easy and efficient. For example, the metallated MOF catalyst could be removed by centrifugation, filtration, a moving bed assembly, or partitioning within the reactor. This easy separation reduces the number of steps necessary to convert olefins to aldehydes or enals.

Metallated MOF catalysts, particularly metallated phosphine MOF catalysts, according to the invention maintain structural integrity following catalysis, which allows for characterization of the catalytic site and surrounding environment, including any potential changes to the MOF structure via SC/PXRD (Single Crystal/Powder X-ray Diffraction), FT-IR (Fourier Transform Infrared Spectroscopy), Raman spectroscopy, XPS (X-ray Photoelectron Spectroscopy) and other known characterization techniques. Metallated MOFs demonstrate steric controlled selectivity of linear/branched aldehyde products and increased aldol condensation products at lower total pressure or for CO-rich syngas mixtures when used in hydroformylation reactions.

Examples of olefins that can be used in accordance with the disclosure herein include arenes and alkenes such as: styrene, cyclobutene, cyclopropene, cyclohexene, cyclooctene, propene, butene, pentene, hexene, heptene and octene. Other examples of olefins that can be used in accordance with the disclosure herein include alpha-olefins, dienes, or polyenes.

HFM reactions using metallated MOF catalysts may be performed at temperatures ranging from 70° C. to 110° C., preferably within a range of 70° C. to 80° C. The pressure maintained for the HFM reactions may preferably range from 40 to 55 bar, most preferably 40 bar.

For example, Table 1 below provides a comparison of Rh-PCM-101 hydroformylation results with substrates including styrene, 1-hexene, and cyclohexene where the partial pressure CO:H₂ ratio is 1:1 and the catalyst loading percentage was determined by ICP-OES (Inductively Coupled Optical-Emission Spectroscopy).

TABLE 1
Comparison of Rh-PCM-101 HFM results with a range of substrates
where the partial pressure ratio of CO:H₂ is 1:1.

| | Catalyst | | | | | | |
|---|---|---|---|---|---|---|---|
| | Loading | | Temper- | Pres- | | | Isomeric |
| | Percent- | Time | ature | sure | Yield | n/ | Product |
| Substrate | age (%) | (h) | (° C.) | (bar) | (%) | iso | (%) |
| Styrene | 0.20 | 20 | 80 | 55 | 90 | 0.4 | N/A |
| 1-hexene | 0.21 | 18 | 80 | 50 | 55 | 4.0 | <1.0 |
| Cyclo- | 0.17 | 18 | 80 | 50 | 85 | N/A | N/A |
| hexene | | | | | | | |

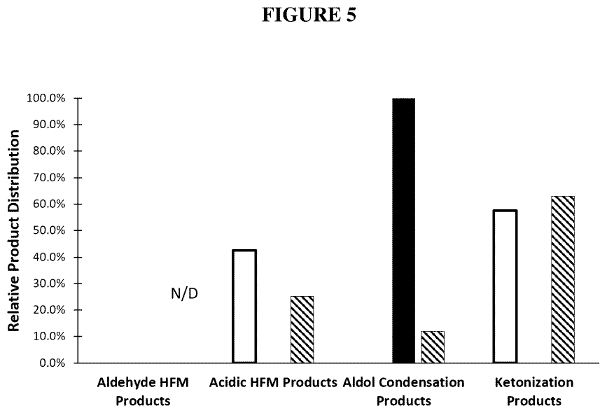

FIG. 5 shows GC-MS (gas chromatography mass spectrometry) product distribution results for the catalysis reactions of 1-hexene at CO:H₂ of 3:1 with Rh-PCM-101 as the catalyst (black bar), n-heptanal at CO:H₂ of 3:1 with Rh-PCM-101 as the catalyst (hatched bars), and n-heptanal at CO:H2 of 3:1 with no Rh-PCM-101 catalyst (white bars). As demonstrated in FIG. 5, use of Rh-PCM-101 as the catalyst in an HFM reaction with 1-hexene as the feedstock and a partial pressure ratio of CO:H₂ of 3:1 results exclusively in aldol condensation products. Alternatively, use of Rh-PCM-101 as the catalyst in an HFM reaction with n-heptanal as the feedstock and a partial pressure ratio of CO:H₂ of 3:1 results in aldol condensation products, ketonization products, and acidic HFM products. When Rh-PCM-101 is not present in the HFM reaction, no aldol condensation products are recovered.

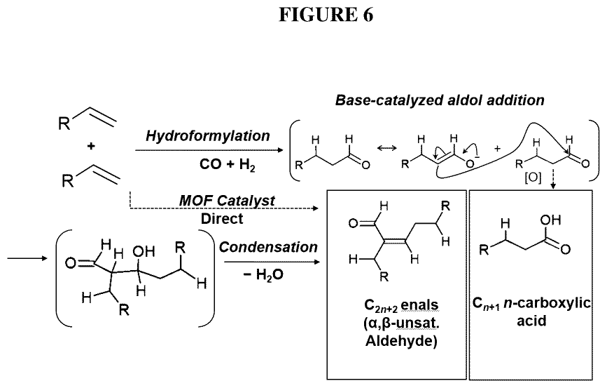

FIG. 6 shows the conversion of olefins to enals and branched alcohols via either the prior art multi-step process or direct tandem catalysis using one of the metallated MOF catalysts described herein. Direct tandem catalysis involves including an acid or base in the same reactor as the olefin feedstock to promote the tandem reaction of HFM followed by aldol condensation. When Rh-PCM-101 is used as a catalyst in a direct tandem HFM/aldol condensation reaction using 1-hexene as the feedstock, the recovered products depend on the partial pressure ratio of CO:H₂ as shown in TABLE 2 below. Specifically, a partial pressure ratio of 1:1 yields Cn+1 n-carboxylic acid products at a yield of 89% and C₂ₙ₊₂ enals at a yield percentage of 11%. When the partial pressure ratio is increased to 3:1, C₂ₙ₊₂ enals are recovered at a yield percentage of 97%. In this fashion, the same metallated MOF catalyst may be used in different HFM/aldol condensation reactions to recover different types of product by altering the pCO:pH₂ ratio.

TABLE 2
Comparison of the effect of the partial pressure ratio
of CO:H2 on products recovered from an HFM reaction

| | | C₂ₙ₊₂ enals (a,b-unsaturated | Cₙ₊₁ n-carboxylic |
|---|---|---|---|
| | | aldehydes) | acid |
| Olefin | pCO:pH₂ | Yield % | Yield % |
| 1-hexene | 1:1 | 11 | 89 |
| 1-hexene | 3:1 | 97 | 3 |

### Arsine-Based MOF Catalysts

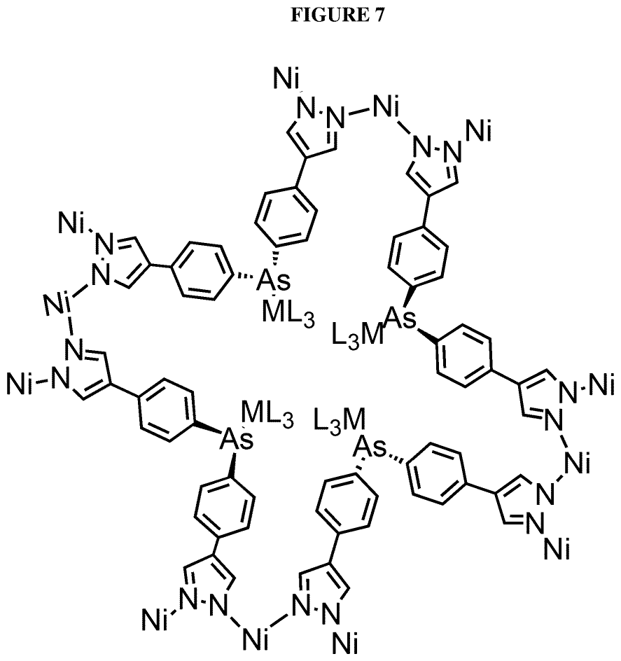

In addition to phosphine-based MOFs, arsine-based MOFs, such as AsCM-102, AsCM-201, or AsCM-303, may be useful as the backbone in metallated MOF catalysts. FIG. 7 shows the structure of AsCM-303 with catalytic sites of metal ligands. 4d and 5d transition metal species are used as the catalyst in a metallated arsine-based MOF catalyst. Preferably, low-valent metal species useful for such catalysts include complexes based on Ru^{II}, Rh^{I}, Ir^{I}, Pd^{II}, and Pt^{II}, bearing common ligands including halides, CO, and/or alkyls. For example, the following are 4d or 5d transition metal species useful to form the arsine-based MOF catalysts disclosed herein: [RhCl(CO)₂]₂, IrCl(C₂H₄)₂, Pt(CH₃)₂Cl₂, Pt(COD)(CH₃)₂, and PdCl₂(COD).

### EXAMPLES

The following examples demonstrate the formation of metallated MOF catalysts, as well as their use in HFM and direct tandem HFM/aldol condensation reactions, including at variable reaction conditions to identify the optimal solvent, temperature, and pressures for specific reaction products. Although the examples provided herein are on a smaller scale than might be required for industrial applications, one of ordinary skill in the art would be able to scale up the conditions indicated to perform reactions on a larger scale.

### Example 1. Rh-PCM-101 Catalyst Formation

To form PCM-101, the MOF-forming linker tris-p-carboxylato(triphenyl)phosphine (tctpH₃) was combined with one equivalent of 4,4′-bipyridine (bipy) and three equivalents of Ni(BF₄)₂·xH₂O in a glass jar. Under an inert N₂ atmosphere, a degassed 5:2:1 mixture of DMF:MeOH:H₂O was added, and the reaction vessel was sealed. The jar was sonicated for 5 minutes to dissolve all solids and then heated at 75° C. for 3 days in a conventional oven. The green crystals of PCM-101(Ni) were then washed with fresh degassed DMF and filtered under vacuum to yield PCM-101, which was stored in air. Additionally, AsCM-101 has been synthesized via the same synthetic procedure, with tris-p-carboxylato(triphenyl)arsine being used in place of the phosphine MOF-forming linker.

To form Rh-PCM-101, under an inert atmosphere, dry crystals of PCM-101 were treated with stoichiometric equivalents of the co-linker 4,4′-bipyridine (bipy) and [RhCl(CO)₂]₂ in a ratio of 1:1:1 (PCM-101:bipy:[RhCl(CO)₂]2) in degassed DMF and heated for 18 hours at 75° C. The flask was swirled periodically throughout the reaction, however not stirred to preserve catalyst crystallinity. Following the reaction, the crystals were cooled to room temperature and washed with degassed DMF and dried under a flow of nitrogen, before being stored under a dry, inert atmosphere. To prepare for catalysis, the solvent in the MOF crystals was removed under vacuum at 80° C. for 18 hours, and subsequently resolvated with the desired chosen catalysis solvent regime.

Although the above example describes how to make Rh-PCM-101, to generate other metallated MOF catalysts based on PCM-101, a different 4d or 5d transition metal species could be substituted for [RhCl(CO)₂]₂ so long as the loading ratio is 1 or below, and preferably, that the stoichiometric equivalent ratio of the MOF backbone, co-linker, and transition metal species is maintained at approximately 1:1:1 with all other conditions kept the same. Examples of those 4d or 5d transition metal species include: IrCl(C₂H₄)₂, Pt(CH₃)₂Cl₂, Pt(COD)(CH₃)₂, and PdCl₂(COD). In addition, PCM-102 or PCM-201 could be substituted for PCM-101 under the same conditions as described above to generate other metallated MOF catalysts based on those MOF backbones.

PCM-201 materials may be formed via coordination of the phosphine MOF-linker (tctpH₃) to a transition metal dimer species prior to MOF synthesis. One example of this is the synthesis of a diosmium material, Os₂-PCM-201. For the synthesis of this material, [Os₂(CO)₆(μ₂-O₂CH)₂] was combined with the phosphine MOF-forming compound, tctpH₃, and heated to reflux in degassed tetrahydrofuran to yield the new MOF-forming diosmium complex, [Os₂(CO)₄(μ₂-O₂CH)₂(tctpH₃)₂]. To form the PCM-201 material, the osmium-phosphine complex was combined with three equivalents of Co(BF₄)₂·xH₂O in a glass jar, a degassed mixture of 5:2:1 DMF:MeOH:H₂O was then added and the reaction vessel was sealed under an inert atmosphere. The vessel was then sonicated for 5 minutes to dissolve all solids, and heated for 2 days at 75° C. in a conventional oven. The formed pink crystals were then washed with degassed DMF and filtered under vacuum to yield the Os₂-PCM-201 material which was stored in air. Under the same procedure, the Os₂-AsCM-201 material can be formed with the arsine compound, tctaH₃, being used in place of the phosphine compound, tctpH₃, to form the MOF-forming arsine-osmium complex, [Os₂(CO)₆(μ₂-O₂CH)₂(tctaH₃)₂].

### Example 2. Solvent Analysis of Rh-PCM-101

3 mg of Rh-PCM-101 was submerged in 50 microliters of 1-octene in a Parr pressure reactor with syngas (CO:H₂ partial pressures at a ratio of 1:1). The temperature was set to 70° C. and the pressure in the reactor set to 40 bar. The same conditions were used for three different reactions, each using 5 ml of a solvent. Toulene, hexane, and DCM were tested. Each reaction was allowed to continue for 15 hours, and the resulting reaction products were characterized as linear, branched-1 or branched-2 products. Table 3 below demonstrates that DCM was the solvent that promoted the best total conversion rate. Also included in Table 3 is the turnover number (TON) and the ratio of linear to branched products (n/iso). The n/iso ratio is particularly important. Typical molecular catalyst-based HFM reactions are much higher than shown in this and the other examples. Metallated MOF catalysts according to the present disclosure demonstrate low n/iso ratios most likely because of the different environment of the catalysts, which are inside micropores that restrict the orientation of the catalyst reaction. This enables a relatively low ratio of linear to branched products.

TABLE 3
Comparison of the effect of solvent on
Rh-PCM-101 catalysis in an HFM reaction

| | | Branched- | Branched- | Total | | |
|---|---|---|---|---|---|---|
| Solvent | Linear | 1 | 2 | Conversion | TON | n/iso |
| Toulene | 50.4% | 42.6% | 8.0% | 65.1% | 150 | 1.0 |
| Hexane | 50.8% | 36.6% | 12.6% | 73.8% | 169 | 1.0 |
| DCM | 57.2% | 33.3% | 9.6% | 84.6% | 202 | 1.3 |

### Example 3. Temperature Analysis of Rh-PCM-101

In each of two reactions, 3 mg of Rh-PCM-101 was submerged in 100 microliters of 1-octene in a Parr pressure reactor with 5 milliliters of toluene, CO:H₂ partial pressures at a ratio of 1:1, and overall pressure set to 40 bar. The temperature was set in one reaction to 70° C. and in a second reaction to 110° C. Each reaction was allowed to continue for 17 hours, and the resulting reaction products were characterized as linear, branched-1 or branched-2 products. Table 4 below demonstrates that the lower temperature resulted in a slightly higher percentage of linear products and lower percentage of branched-1 products, all other variables being held equal. Temperature appeared to have no effect on the percentage of branched-2 products under these conditions. Also included in Table 4 is the turnover number (TON) and the ratio of linear to branched products (n/iso).

TABLE 4
Comparison of the effect of temperature on
Rh-PCM-101 catalysis in an HFM reaction

| Temper- | | Branched- | Branched- | Total | | |
|---|---|---|---|---|---|---|
| ature | Linear | 1 | 2 | Conversion | TON | n/iso |
| 70° C. | 52.05% | 36.71% | 11.24% | 33.40% | 67 | 1.09 |
| 110° C. | 46.04% | 42.66% | 11.30% | 27.55% | 41 | 0.90 |

### Example 4. Catalyst Stability and Reusability Analysis of Rh-PCM-101

3 mg of Rh-PCM-101 was submerged in 100 microliters of 1-octene in a Parr pressure reactor with 5 milliliters of toluene, CO:H₂ partial pressures at a ratio of 1:1, temperature set to 70° C., and overall pressure set to 40 bar. To evaluate catalyst stability and reusability, the resulting reaction products were characterized after 1, 2, and 3 cycles of catalysis, with each cycle lasting 15 hours. Table 5 below demonstrates that a only small amount of total conversion percentage of Rh-PCM-101 is lost after the first cycle, and it remains relatively stable over multiple cycles of catalysis.

TABLE 5
Comparison of the effect of repeated cycles
on Rh-PCM-101 catalysis in an HFM reaction

| | | Branched- | Branched- | Total | | |
|---|---|---|---|---|---|---|
| Catalyst | Linear | 1 | 2 | Conversion | TON | n/iso |
| Cycle-1 | 52.05% | 36.71% | 11.24% | 33.40% | 67 | 1.09 |
| Cycle-2 | 46.08% | 40.86% | 13.06% | 29.70% | 50 | 0.85 |
| Cycle-3 | 39.80% | 39.80% | 13.02% | 28.90% | 53 | 0.89 |

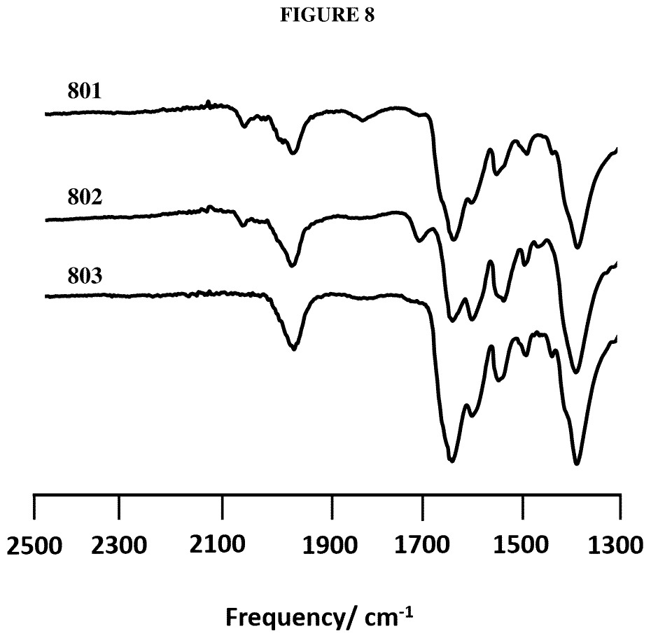

Further analysis was done to look at the structure of Rh-PCM-101 before and after catalysis. FIG. 8 shows the FT-IR spectra of Rh-PCM-101 charged with CO (801), Rh-PCM-101 post-catalysis (802), and Rh-PCM-101 (803). This FT-IR spectra of Rh-PCM-101 before and after an HFM reaction shows new Rh-CO environments formed, that may also be formed by charging the Rh-PCM-101 with CO gas.

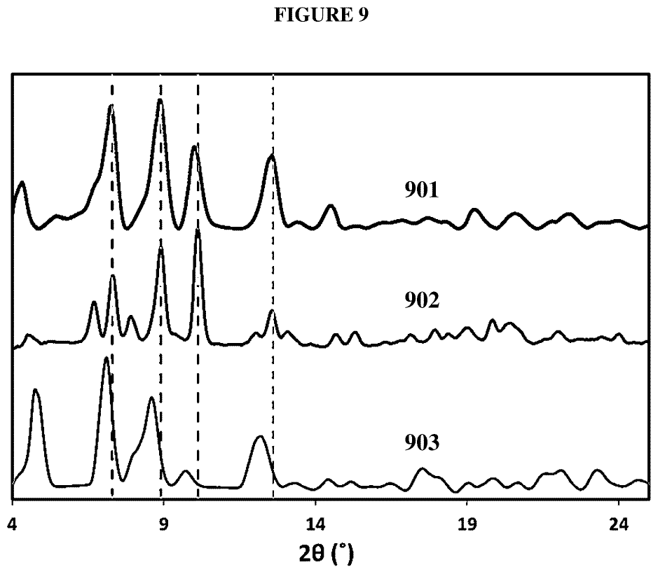

FIG. 9 shows PXRD results for Rh-PCM-101 post-catalysis (901), Rh-CO-PCM-101 (902), and PCM-101 (903). These results demonstrate the stability of the PCM-101 backbone structure both after metalation with Rh and charging with CO gas and after catalysis.

### Example 5. Rh-AsCM-102 Catalyst Formation

To form AsCM-102, the MOF-forming linker tris-p-carboxylato(triphenyl)arsine (tctaH₃) was combined with one equivalent of 4,4′-bipyridine (bipy), three equivalents of Co(BF₄)₂·xH₂O, and twenty equivalents of benzoic acid into a glass jar. Under an inert N₂ atmosphere, a degassed 5:2:1 mixture of DMF:MeOH:H₂O was added, and the reaction vessel was sealed. The jar was sonicated for 5 minutes to dissolve all solids and then heated at 75° C. for 2 days in a conventional oven. The pink crystals of AsCM-102(Co) were then washed with fresh degassed DMF and filtered under vacuum to yield the AsCM-102 which was stored in air. Alternatively, PCM-102 has been synthesized via the same synthetic procedure, with tris-p-carboxylato(triphenyl)phosphine used in place of the arsine MOF-forming linker.

To form Rh-AsCM-102, under an inert atmosphere, AsCM-102 was treated with a half equivalent of [RhCl(CO)₂]₂ in a degassed 1:1 DMF:DCM mixture and heated for 18 hours at 75° C. In other words, the stoichiometric equivalent ratio of [RhCl(CO)₂]₂ to AsCM-102 was 1:2. The flask was swirled periodically throughout the reaction, however it was not stirred to preserve catalyst crystallinity. Following the reaction, the crystals were cooled to room temperature and washed with degassed DMF, dried under a flow of nitrogen, before being stored under a dry, inert atmosphere. To prepare for catalysis, the solvent in the MOF crystals was removed under vacuum at 80° C. for 18 hours, and subsequently resolvated with the desired chosen catalysis solvent regime.

Although the above example describes how to make Rh-AsCM-102, to generate other metallated MOF catalysts based on AsCM-102, a different 4d or 5d transition metal species could be substituted for [RhCl(CO)₂]₂ so long as the loading ratio is 1 or below, and preferably, that the stoichiometric equivalent ratio of the transition metal species to MOF backbone is maintained at approximately 1:2 with all other conditions kept the same. Examples of those 4d or 5d transition metal species include: IrCl(C₂H₄)₂, Pt(CH₃)₂Cl₂, Pt(COD)(CH₃)₂, and PdCl₂(COD). In addition, AsCM-101 or AsCM-303 could be substituted for AsCM-102 under the same conditions as described above to generate other metallated MOF catalysts based on those MOF backbones.

### Example 6. Solvent Analysis of Rh-AsCM-102

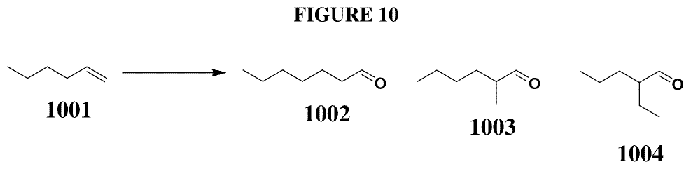

FIG. 10 shows an HMF reaction of 1-hexene (1001), which may yield various products depending on the conditions including n-heptanal (1002, linear), 2-methyl-hexanal (1003, branched-1), and 2-ethyl-pentanal (1004, branched-2). To evaluate the optimal solvent for this reaction, 20 microliters of 1-hexene were reacted in a Parr pressure reactor with 5 mg of Rh-AsCM-102-Co, 5 ml of solvent, a partial pressure ratio for CO:H2 of 1:1 and total pressure of 40 bar for seventeen hours at 70° C. Solvents tested included toluene, hexane, and THF. Characterization of the resulting reaction products indicated that hexane was optimal for the total conversion rate. Table 6 provides characterization of the reaction products for each solvent tested, as well as the TON, TOF/h (turnover frequency per hour), and n/iso.

TABLE 6
Comparison of the effect of solvent on Rh-
AsCM-102 catalysis in an HFM reaction

| | | Branched- | Branched- | Total | | |
|---|---|---|---|---|---|---|
| Solvent | Linear | 1 | 2 | Conversion | TON | n/iso |
| Toulene | 41.29% | 49.25% | 9.46% | 29.9% | 121.04 | 0.70 |
| Hexane | 41.40% | 54.74% | 3.86% | 95.16% | 385.25 | 0.71 |
| THF | 39.69% | 49.59% | 10.72% | 60.46% | 244.75 | 0.66 |

### Example 7. Temperature Analysis of Rh-AsCM-102

In each of three reactions, 5 mg of Rh-AsCM-102-Co was submerged in 20 microliters of 1-hexene in a Parr pressure reactor with 5 milliliters of hexane, CO:H₂ partial pressures at a ratio of 1:1, and overall pressure set to 40 bar. The temperature was set in one reaction to 50° C., in a second reaction to 70° C., and in a third reaction to 100° C. Each reaction was allowed to continue for 17 hours, and the resulting reaction products were characterized as linear, branched-1 or branched-2 products. Table 7 below demonstrates that the 70° C. resulted in a higher percentage total conversation percentage. Also included in Table 7 is the TOF, turnover number (TON)/h, and the ratio of linear to branched products (n/iso).

TABLE 7
Comparison of the effect of temperature on
Rh-AsCM-102 catalysis in an HFM reaction

| | | | | Total | | | |
|---|---|---|---|---|---|---|---|
| Temper- | | Branch- | Branch- | Conver- | | | |
| ature | Linear | ed-1 | ed-2 | sion | TOF | TON | n/iso |
| 50° C. | 41.44% | 53.17% | 5.38% | 79.41% | 321.47 | 21.43 | 0.71 |
| 70° C. | 41.40% | 54.74% | 3.86% | 95.16% | 385.25 | 25.68 | 0.71 |
| 100° C. | 50.10% | 50.10% | 3.53% | 90.45% | 366.18 | 24.41 | 0.86 |

### Example 8. Time Analysis of Rh-AsCM-102 Catalysis

The impact of reaction time on Rh-AsCM-102 was also investigated. Six independent HFM reactions were run where the only changed variable was the time of the reaction cycle. Reactions proceeded for 1 hour, 3 hours, 5 hours, 10 hours, 17 hours, and 24 hours. Each reaction otherwise had the following conditions: 20 microliters 1-hexene, 5 milligrams Rh-AsCM-102, partial pressure ratio CO:H₂ of 1:1, overall pressure of 40 bar, 5 ml of hexane as the solvent and 70° C. Table 8 presents the results of this experiment and demonstrates that the total conversion percentage jumped significantly from 10 hours to 17 hours and by 24 hours, the turnover frequency had been significantly reduced.

TABLE 8
Comparison of the effect of reaction time
on Rh-AsCM-102 catalysis in an HFM reaction

| | | | | Total | | | |
|---|---|---|---|---|---|---|---|
| | | Branched- | Branched- | Conver- | | TOF | |
| Time | Linear | 1 | 2 | sion | TON | (h⁻¹) | n/iso |
| 1 h | 43.1 | 50.6 | 6.3 | 26.6% | 107.6 | 107.6 | 0.76 |
| 3 h | 43.3 | 51.6 | 5.2 | 38.4% | 155.4 | 23.9 | 0.77 |
| 5 h | 43.5 | 51.6 | 4.9 | 41.5% | 168.0 | 6.3 | 0.77 |
| 10 h | 41.6 | 54.0 | 4.4 | 52.4% | 212.0 | 8.8 | 0.71 |
| 17 h | 41.4 | 54.7 | 3.9 | 95.2% | 385.3 | 24.8 | 0.71 |
| 24 h | 41.2 | 48.5 | 3.8 | 95.6% | 386.9 | 1.6 | 0.70 |

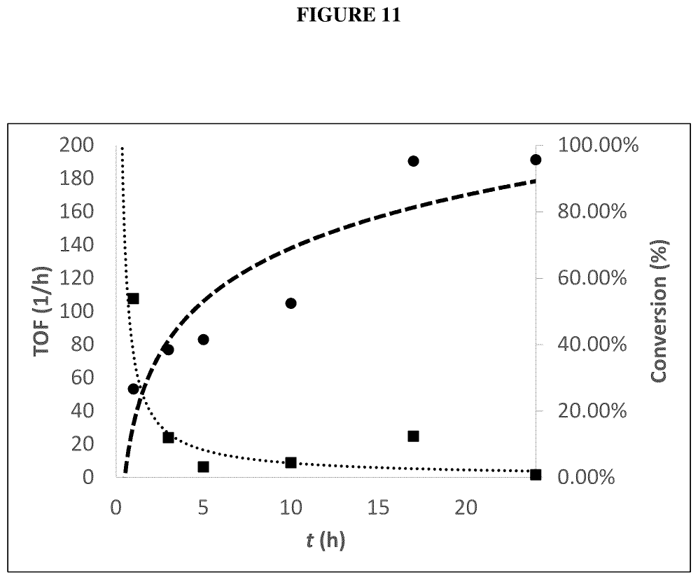

FIG. 11 shows a graph with conversion percentage and TOF (turnover frequency) plotted against time for the HMF reactions of 1-Hexene using Rh-AsCM-102 as the catalyst at a partial pressure ratio of 1:1 for CO:H₂, 40 bar pressure, and 70° C.

### Example 9. Catalyst Stability and Reusability Analysis of Rh-AsCM-102

5 mg of Rh-AsCM-102-Co was submerged in 20 microliters of 1-hexene in a Parr pressure reactor with 5 milliliters of hexane, CO:H₂ partial pressures at a ratio of 1:1, temperature set to 70° C., and overall pressure set to 40 bar. To evaluate catalyst stability and reusability, the resulting reaction products were characterized after 1, 2, 3, 4, and 5 cycles of catalysis, with each cycle lasting 17 hours. Table 9 below demonstrates that the highest total conversion rate for Rh-AsCm-102 is seen after the first cycle, but it remains an effective catalyst through multiple cycles.

TABLE 9
Comparison of the effect of repeated cycles
on Rh-AsCM-102 catalysis in an HFM reaction

| | | Branched- | Branched- | Total | | n/ |
|---|---|---|---|---|---|---|
| Catalyst | Linear | 1 | 2 | Conversion | TON | iso |
| Cycle-1 | 41.40% | 54.74% | 3.86% | 95.16% | 385.25 | 0.71 |
| Cycle-2 | 41.66% | 54.15% | 4.19% | 92.04% | 372.63 | 0.71 |
| Cycle-3 | 44.44% | 52.22% | 3.34% | 85.79% | 347.30 | 0.80 |
| Cycle-4 | 43.71% | 51.36% | 4.93% | 78.46% | 317.64 | 0.78 |
| Cycle-5 | 45.27% | 49.46% | 5.28% | 62.60% | 253.44 | 0.83 |

PXRD analysis of Rh-AsCM-102 comparing AsCM-102, Rh-AsCM-102, and Rh-AsCM-102 after three cycles of catalysis indicated that the structure of AsCM-102 remains relatively stable when metallated with Rh and after multiple rounds of catalysis.

### Example 10. Analysis of Metallated MOF Catalysts in an HFM Reaction

Multiple metallated MOF catalysts were analyzed in HFM reactions using 1-hexene as the feedstock. Specifically, Rh-AsCM-102, Rh-PCM-102, and a combination containing a material where phosphine and arsine sites are mixed in a 1:1 ratio within a single crystal, Rh-PCM-102:Rh-AsCM-102, were evaluated. In each reaction, 20 microliters of 1-hexene and 5 mg of the catalyst being tested were used in an HFM reaction in a Parr pressure reactor. For each reaction, temperature was set to 70° C., the partial pressure ratio of CO:H₂ was 1:1, and the total overall pressure was maintained at 40 bar. Table 10 presents the results of those reactions and demonstrates that Rh-AsCM-102 performed the best in terms of total conversion percentage under these conditions.

TABLE 10
Comparison of the effect of the catalyst on HFM reactions

| | | | | Total | | |
|---|---|---|---|---|---|---|
| | | Branch- | Branch- | Conver- | | n/ |
| Catalyst | Linear | ed-1 | ed-2 | sion | TON | iso |
| Rh-AsCM-102 | 41.40% | 54.74% | 3.86% | 95.16% | 385.25 | 0.71 |
| Rh-PCM-102 | 41.44% | 53.17% | 5.38% | 79.41% | 321.47 | 0.71 |
| Rh-PCM- | 45.70% | 49.34% | 4.96% | 80.16% | 324.51 | 0.84 |
| 102:Rh- | | | | | | |
| AsCM-102 | | | | | | |
| (1:1) | | | | | | |

### Example 11. Hydroformylation of Hexene Using Rh-AsCM-102, Followed by Enolization of Heptanal

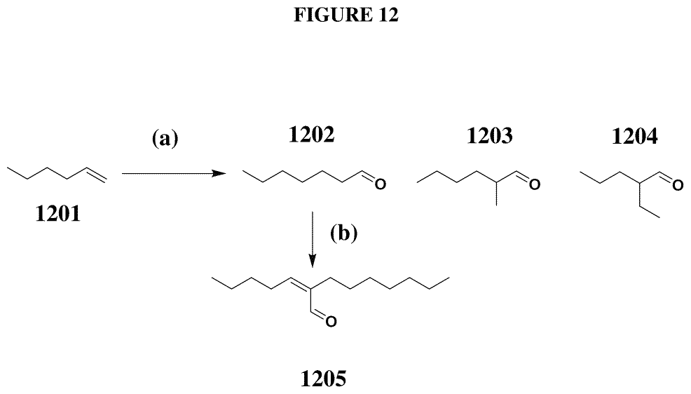

FIG. 12 shows (a) an HFM reaction of 1-hexene (1201) using Rh-AsCM-102 as the catalyst to yield n-heptanal (1202) as the major product with 2-methyl-hexanal (1203) and 2-ethyl pentanal (1204) as minor products, (b) followed by enolization of the heptanal to yield (Z)-2-pentylidenenonanal (1205). Each HFM reaction used 20 microliters 1-hexene, 5 mg Rh-AsCM-102, partial pressure ratio CO:H2 of 1:1, overall pressure of 40 bar, 5 ml hexane as the solvent and continued for 17 hours at 70° C. The Rh-AsCM-102 was then removed from the reactor, and one of the following added at a concentration of 0.01M to the direct liquid products of the HFM reaction: aqueous sodium hydroxide, t-BuOK, or ZSM-5. The aqueous NaOH is an inorganic base, the t-BuOK acts as an organic base, and ZSM-5 is a Bronsted acid. Each enolization reaction was allowed to proceed for 17 hours at 70° C. with a partial pressure ratio CO:H₂ of 1:1 and overall pressure of 40 bar. Each reaction (whether using aqueous sodium hydroxide, t-BuOK, or ZSM-5) demonstrated the quantitative conversion of the linear product from the HFM reaction into an enal, specifically, the enolization of the heptanal to yield (Z)-2-pentylidenenonanal.

1. A catalyst comprised of:
a. a metal-organic framework; and
b. a 4d or 5d transition metal species contained in one or more active sites, wherein the catalyst is a microporous solid state ligand.

2. The catalyst of claim 1 wherein the metal-organic framework is a phosphine-based metal organic framework.

3. The catalyst of claim 2 wherein the metal-organic framework is PCM-101, PCM-102, or PCM-201.

4. The catalyst of claim 1 wherein the metal-organic framework is an arsine-based metal organic framework.

5. The catalyst of claim 4 wherein the arsine-based metal organic framework is AsCM-102, AsCM-201, or AsCM-303.

6. The catalyst of claim 1 wherein the 4d or 5d transition metal species is one of Ru^{II}, Rh^{I}, Ir^{I}, Os, Pd^{II}, and Pt^{II} bearing common ligands.

7. The catalyst of claim 1 wherein the metal-organic framework is PCM-101 and the 4d or 5d transition metal species is a Rh^{I} bearing common ligand.

8. The catalyst of claim 1 wherein the metal-organic framework is PCM-101 and the 4d or 5d transition metal species is a Ir^{I} bearing common ligand.

9. The catalyst of claim 1 wherein the metal-organic framework is PCM-101 and the 4d or 5d transition metal species is a Pt^{II} bearing common ligand.

10. The catalyst of claim 1 wherein the metal-organic framework is AsCM-102 and the 4d or 5d transition metal species is a Rh^{I} bearing common ligand.

11. A catalysis method comprising:
a. submerging a metallated MOF catalyst in a liquid solvent inside a reactor;
b. adding an olefin to the reactor;
c. maintaining a CO:H₂ partial pressure ratio in the reactor within a range of 1:1 and 3:1;
d. maintaining the temperature of the reactor between 55° C. and 85° C. for a reaction time;
e. maintaining the overall pressure in the reactor between 40 and 55 bar during the reaction time;
wherein the reaction time is between eleven and twenty-four hours.

12. The method of claim 11 further comprising (f) removing the metallated MOF catalyst; (g) adding one or more of aqueous sodium hydroxide, an organic base, or a Bronsted acid to the reactor; and (h) recovering an enal product.

13. The method of claim 11 wherein the metallated MOF catalyst is one of Rh-PCM-101, Ir-PCM-101, Pt-PCM-101, Rh-PCM-102, Ir-PCM-102, Pt-PCM-102, Rh-PCM-201, Ir-PCM-201, Pt-PCM-201, Rh-AsCM-102, Rh-AsCM-201, Rh-AsCM-303, Ir-AsCM-102, Ir-AsCM-201, Ir-AsCM-303, Pt-AsCM-102, Pt-AsCM-201, or Pt-AsCM-303.

14. The method of claim 11 wherein the olefin is one of: styrene, cyclobutene, cyclopropene, cyclohexene, cyclooctene, propene, butene, pentene, hexene, heptene and octene.

15. The method of claim 12 wherein the metallated MOF catalyst is Rh-PCM-101, the olefin is propene, and the recovered enal product is 2-ethyl-2-hexenal.

16. The method of claim 12 wherein the metallated MOF catalyst is Rh-AsCM-102, the olefin is propene, and the recovered enal product is 2-ethyl-2-hexenal.

17. The method of claim 12 wherein the CO:H₂ partial pressure ratio is 1:1.

18. The method of claim 12 wherein removing the metallated MOF catalyst comprises filtering out the metallated MOF catalyst.

19. The method of claim 12 wherein the reactor is a moving bed reactor and removing the metallated MOF catalyst comprises using a moving bed assembly to remove the metallated MOF catalyst from one or more reactants before steps (g) and (h) are performed.

20. The method of claim 12 wherein removing the metallated MOF catalyst comprises partitioning the metallated MOF catalyst away from one or more reactants before steps (g) and (h) are performed.
